(19) United States
(12) Patent Application Publication
(10) Pub. No.: US 2018/0099163 A1
(43) Pub. Date: Apr. 12, 2018

(54) APPARATUS AND METHOD FOR DAMAGING OR DESTROYING ADIPOCYTES

(71) Applicant: Mattioli Engineering Corporation, McLean, VA (US)

(72) Inventor: Gian Franco BERNABEI, Florence (IT)

(73) Assignee: Mattioli Engineering Corporation, McLean, VA (US)

(21) Appl. No.: 15/837,821

(22) Filed: Dec. 11, 2017

Related U.S. Application Data
(63) Continuation-in-part of application No. 14/739,040, filed on Jun. 15, 2015.

(51) Int. Cl. A61N 7/00

(52) U.S. Cl. CPC A61N 7/00; A61N 2007/0008

(57) ABSTRACT

An apparatus for treating adipose tissue located beneath a patient's skin includes a main body portion that houses an ultrasound transducer that outputs transverse ultrasound waves to be applied to a patient's skin. The apparatus also includes a semisphere portion provided at a distal end of the apparatus and that is configured to contact the patient's skin when the patient is being treated with the apparatus. The apparatus further includes an intermediate portion provided between the main body portion and the semisphere portion, in which a connector that provides the ultrasound waves from the ultrasound transducer to the semisphere portion is housed. The intermediate portion is disposed substantially perpendicular to the main body portion. An embodiment composed by two apparatus connected back to back in order to eliminate vibrations and emission of longitudinal waves in direction perpendicular to the skin.

### CROSS REFERENCE TO RELATED APPLICATIONS

This application is a continuation in part of application Ser. No. 14/739,040, filed on Jun. 15, 2015, entitled “APPARATUS AND METHOD FOR DAMAGING OR DESTROYING ADIPOCYTES”, the entire contents of all of which are incorporated herein by reference.

### FIELD OF THE INVENTION

This invention relates to an apparatus and method for applying transversal ultrasound waves to a patient's skin in order to damage and/or destroy adipocytes and fatty deposits located under the dermis of the patient's skin while avoiding the generation of longitudinal ultrasound waves perpendicular to the skin surface beyond a particular depth.

### BACKGROUND

Procedures currently exist for removing fat cells under the skin, whereby those fat cells or adipose cells are also commonly referred to as “adipocytes.” One such procedure ruptures adipocytes using longitudinal ultrasound waves, whereby ultrasound waves are applied to adipose tissue beneath the skin surface (the dermis). The ultrasound waves rupture the adipocytes in the adipose tissue under the skin surface, causing necrosis, which can cause extensive collateral damage to other non-fat tissue (e.g., blood vessels, connective tissue, dermis, etc.).

U.S. Pat. No. 8,579,835 to Britva et al. describes an improved fat cell killing apparatus and method, which applies both transverse ultrasound waves and longitudinal ultrasound waves to a patient's skin. Britva's uses a sonotrobe to apply the transverse ultrasound waves and longitudinal ultrasound waves to a patient's skin, whereby the sonotrobe has a curved distal portion having a plurality of undulations or ridges provided along the curved distal portion, for application of transverse ultrasound waves to a patient's skin.

Britva describes applying longitudinal ultrasound waves to a patient's skin during a hot mode of operation, and to apply transverse ultrasound waves to the patient's skin during a cold mode of operation, in order to enhance the destruction of adipocytes under the skin surface. Britva describes that adipocytes typically die within three days after treatment of a patient's skin with both transverse ultrasound waves and longitudinal ultrasound waves.

Britva describes the use of two resonant frequencies: a) a cold mode resonant frequency of about 69 kHz, and b) a hot mode resonant frequency of about 60 kHz. During operation in the cold mode, Britva's sonotrobe applies ultrasound vibrations in the distal portion of his sonotrobe primarily in a direction substantially perpendicular to the elongate neck axis (e.g., the longitudinal axis) of the sonotrobe, and whereby a transverse mechanical standing wave is generated in the distal portion of his sonotrobe by way of ridges that convert longitudinal waves to transverse waves, for application to the patient's skin. During operation in the hot mode, Britva's sonotrobe applies ultrasound vibrations in the distal portion of his sonotrobe primarily in a direction substantially parallel to the elongate neck axis of the sonotrobe, and whereby a longitudinal mechanical standing wave is generated in the distal portion of his sonotrobe.

Britva goes on to describe that application of ultrasound waves in the cold mode and the hot mode provides for good results regarding destruction of adipocytes under the skin surface.

Britva's ultrasound generator housed within a proximal part of his sonotrobe only outputs longitudinal waves, for which some of those waves are converted to transverse waves by way of the complex structure of his distal curved portion with plural ridges or undulations (as shown in FIGS. 9A-9C of Britva).

### SUMMARY

It is known in the art that the low frequency longitudinal waves have a high depth of penetration inside the human body. For this reason low frequency longitudinal waves are considered safe only frequency above 50 KHz because higher attenuation occurs in human tissue in these frequency ranges. Low frequency longitudinal waves are recognized as good choice in order to damage the adipocytes. But several complex technical challenges, like focusing, need to be overcome in order to avoid the undesirable application of high energy levels being inside the inner organs of the body. Moreover it is well known that with low frequency longitudinal waves, is very easy to generate cavitation, which, while dissolving the fatty deposits, may cause damage to human tissue. Instead, low frequency ultrasound waves should be used where very high energy is concentrated in the first two centimeters of depth and high attenuation is experienced beyond the first two centimeters. Moreover, at a frequency of 30 KHz, due to the low propagation speed, around 5 meters per seconds, the wavelength of the transverse ultrasound waves is around 160 micron, which is the same order of magnitude of the resonant frequency of adipocytes. An adipocyte treated under the transverse ultrasound waves at a frequency of around 30 KHz will resonate and the fatty deposit inside is damaged. The generation of low frequency transversal ultrasound waves usually causes also the generation of longitudinal waves at the same time. The purpose of this invention is to avoid the generation of longitudinal waves that could reach inner part of the body in direction perpendicular to the skin surface or limit their intensity.

One innovative aspect of the subject matter described in this specification can be embodied in an apparatus for treating adipose tissue located beneath a patient's skin. The apparatus includes a main body portion that houses an ultrasound transducer, in which the main body portion is provided at a proximal end of the apparatus furthest from the patient's skin when the patient is being treated with the apparatus. The apparatus further includes a semisphere portion provided at a distal end of the apparatus and that is configured to contact the patient's skin when the patient is being treated with the apparatus. The apparatus further includes an intermediate portion provided between the main body portion and the semisphere portion, in which the intermediate portion is disposed substantially perpendicular to the main body portion such that a main axis of the main body portion is provided along a first plane substantially parallel to a second plane corresponding to a surface of the patient's skin being treated with the apparatus, and such that a main axis of the intermediate portion is provided along a third plane substantially perpendicular to the second plane. The ultrasound transducer is configured to vibrate along the first plane and to thereby cause the semisphere portion to vibrate substantially parallel to the patient's skin due to a connector provided within the intermediate portion that connects the ultrasound transducer to the semisphere. This results in transverse ultrasound waves being applied to the patient's skin by way of the apparatus, which results in destruction and/or damage to adipocytes located beneath a dermis of the patient's skin.

Another innovative aspect of the subject matter described in this specification can be embodied in a method for treating adipose tissue located beneath a patient's skin. The method includes outputting transverse ultrasound vibrations from an ultrasound transducer provided in a proximal end of the probe to a first end of a connecting rod. The method also includes providing the transverse ultrasound vibrations from the first end of the connecting rod to a second end of the connecting rod that is connected to a semisphere portion located at a distal end of the probe. The transverse ultrasound vibrations are configured to be applied to the patient's skin by contacting the semisphere portion of the probe to the patient's skin.

In one or more additional embodiments, an apparatus having two semisphere portions are provided to provide a symmetrical device, which overcomes potential drawbacks to non-symmetrical or non-complimentary transducers in which small ultrasonic vibrations in direction perpendicular to the skin may be disadvantageously generated if the device is not well balanced.

For example, in accordance with one or more embodiments, an apparatus may be provided for treating adipose tissue located beneath a skin surface of a patient. The apparatus may comprise a main body portion that houses a first ultrasound transducer positioned in such a way in order to vibrate only in a first direction parallel to the skin surface; a first semispherical portion provided at first distal end of the apparatus and configured to be pressed against the skin in order to bend the skin in such a way that a lower portion of the first semispherical portion is positioned at a same depth as a surrounding first layer of adipocytes to be treated; and a first rigid intermediate portion provided between the first ultrasound transducer and the first semispherical portion, the first rigid intermediate portion coupling vibration from the first ultrasound transducer to the first semispherical portion to cause the first semispherical portion to vibrate only in the first direction, with a same intensity and with a same phase of the first ultrasound transducer and only parallel to the skin surface and without vibration in a direction perpendicular to skin surface, the coupled vibration generating transverse ultrasound waves perpendicular to the skin surface and at a predetermined depth, and longitudinal waves in the first direction parallel to the skin surface. In one or more embodiments, a cross-sectional curvature radius of a distal end of the first semispherical portion may be set to establish a depth at which adipose cells in the adipose tissue located beneath the skin surface of the patient are damaged or destroyed when the first semispherical portion is pressed against the skin surface of the patient to the established depth. In one or more embodiments, the cross-sectional curvature radius of the distal end of the first semispherical portion may further be set such that a maximum intensity of the of the transverse ultrasound waves occurs at the established depth.

In one or more embodiments, the first semispherical portion has a smooth outer surface to facilitate movement on the skin surface during pressing. In one or more embodiments, a frequency of the transverse ultrasound vibrations are within the range of from 20 kHz-60 kHz. In one or more embodiments, the transverse ultrasound vibrations are pulsed within a range of 50 Hz-100 Hz and the duty cycle between 10% and 50%. In one or more embodiments, the first semispherical portion may be configured to press into the surface of the skin at a depth of between 5 mm to 40 mm thus generating transverse ultrasound waves at the corresponding depth. In accordance with one or more embodiments, a diameter of the first semispherical portion may be approximately 50 mm. In accordance with one or more embodiments, the power flux applied to patient skin is in a range if between 1 W/cm² to 3 W/cm². In one or more embodiments, the intermediate portion between the transducer and the semispherical may be made of aluminum. In one or more additional or alternative embodiments, the intermediate portion between the transducer and the semispherical may be made of steel.

In still further accordance with one or more embodiments, an exemplary apparatus may further comprise a second ultrasound transducer housed in the main body portion and positioned next to and in an opposing driving relation to the first ultrasound transducer in such a way as to vibrate only in a second direction parallel to the skin surface and opposite to the first direction; a second semispherical portion provided at the distal end of the apparatus and configured to be pressed against the skin in order to bend the skin in such a way that a lower portion of the second semispherical portion is positioned at the same depth as the surrounding first layer of the adipocytes to be treated; and a second rigid intermediate portion provided between the second ultrasound transducer and the second semispherical portion, the second rigid intermediate portion coupling vibration from the second ultrasound transducer to the second semispherical portion to cause the second semispherical portion to vibrate only in a same direction, with a same intensity and with a same phase of the second ultrasound transducer; in an opposite direction, with a same intensity and with an opposite phase of the first ultrasound transducer and only parallel to the skin surface and without vibration in the direction perpendicular to the skin surface, the coupled vibration generating transverse ultrasound waves perpendicular to the skin surface and at the predetermined depth, and longitudinal waves in the direction parallel to the skin surface. In one or more embodiments, a cross-sectional curvature radius of a distal end of the second semispherical portion is set at the depth at which adipose cells in the adipose tissue located beneath the skin surface of the patient are damaged or destroyed when the second semispherical portion is pressed against the skin surface of the patient to the established depth. In one or more embodiments, the cross-sectional curvature radius of the distal end of the second semispherical portion is further set such that the maximum intensity of the transverse ultrasound waves occurs at the established depth.

### BRIEF DESCRIPTION OF THE DRAWINGS

The details of one or more embodiments of the subject matter described in this specification are set forth in the accompanying drawings and the description below. Other features, aspects, and advantages of the subject matter will become apparent from the description, the drawings, and the claims.

FIG. 1 shows a probe for applying transverse ultrasound waves to a patient's skin according to one or more embodiments;

FIG. 2 shows elements of a probe for applying transverse ultrasound waves, such as a probe of FIG. 1, in exploded view;

FIG. 3 shows an exemplary configuration of an ultrasound transducer provided in a probe for applying transverse ultrasound waves, such as a probe of FIG. 1, according to one or more embodiments;

FIG. 4 shows a probe for applying transverse ultrasound waves, such as a probe of FIG. 1, to a patient's skin, in order to destroy or damage adipose cells under the patient's skin, according to one or more embodiments;

FIG. 5 shows components housed within a probe for applying transverse ultrasound waves, such as a probe of FIG. 1, that may be used to create transverse ultrasound vibrations and apply those vibrations to the patient's skin, according to one or more embodiments;

FIGS. 6A and 6B show the semisphere portion of the probe of FIG. 1, in a front view and a side view, respectively;

FIG. 7 shows an exemplary configuration in which two semisphere portions are configured to ensure that the emission of longitudinal ultrasound waves perpendicular to the patient's skin is at or near zero; and

FIG. 8 shows elements of a probe for applying transverse ultrasound waves, such as a probe of FIG. 7 in an exploded view.

Like reference numbers and designations in the various drawings indicate like elements.

### DETAILED DESCRIPTION

The present specification is directed to an apparatus and method for applying transversal ultrasound waves to a patient's skin in order to damage and/or destroy adipocytes located under the dermis of the patient's skin in which two semisphere portions are provided.

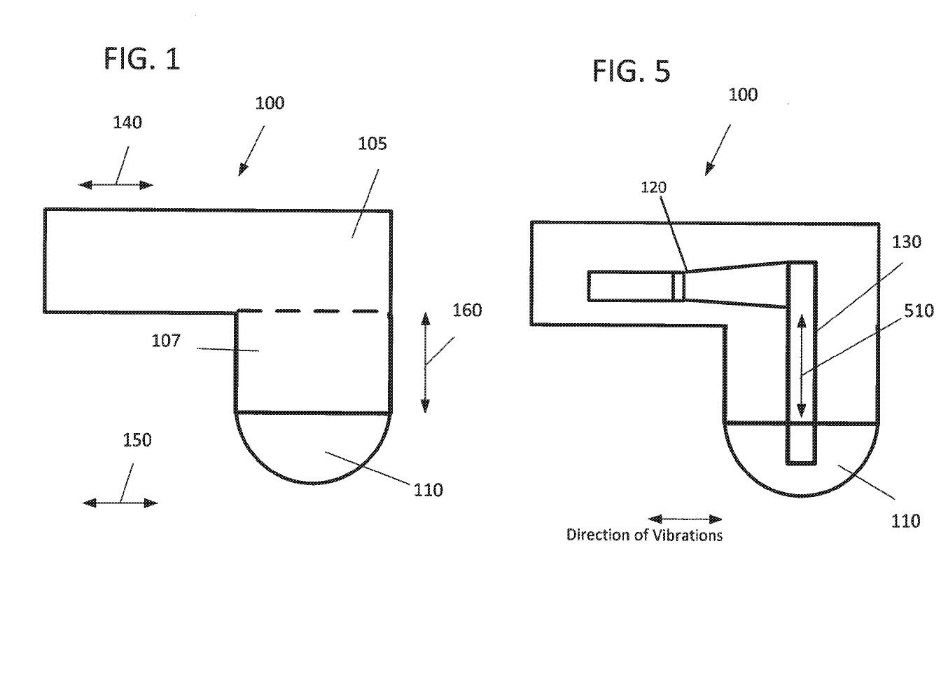

According to one or more embodiments, a probe provides transverse ultrasound vibrations to a patient's skin, whereby those transverse ultrasound vibrations impinge on the skin surface substantially parallel to the skin surface, and enter into the skin surface a predetermined depth, such as 20-40 mm before being substantially attenuated, so as to damage and/or destroy adipose cells within a certain range (e.g., 0.01 to 40 mm) under the skin surface. FIG. 1 shows a probe 100 according to one or more embodiments of the invention.

Referring now to FIG. 1 and FIG. 5, a distal end of the probe 100 includes a semisphere portion 110, whereby the semisphere portion 110 receives transverse ultrasound vibrations output from an ultrasound transducer 120, provided in a main body portion 105 of the probe 100 (that is provided at a proximal end of the probe 100). A metal component, also referred herein as a connector rod 130, is provided within an intermediate portion 107 of the probe 100, whereby the connector rod 130 transfers transverse ultrasound vibrations output from the ultrasound transducer 120 directly to the semisphere portion 110 of the probe, for application of those transverse ultrasound vibrations to the patient's skin.

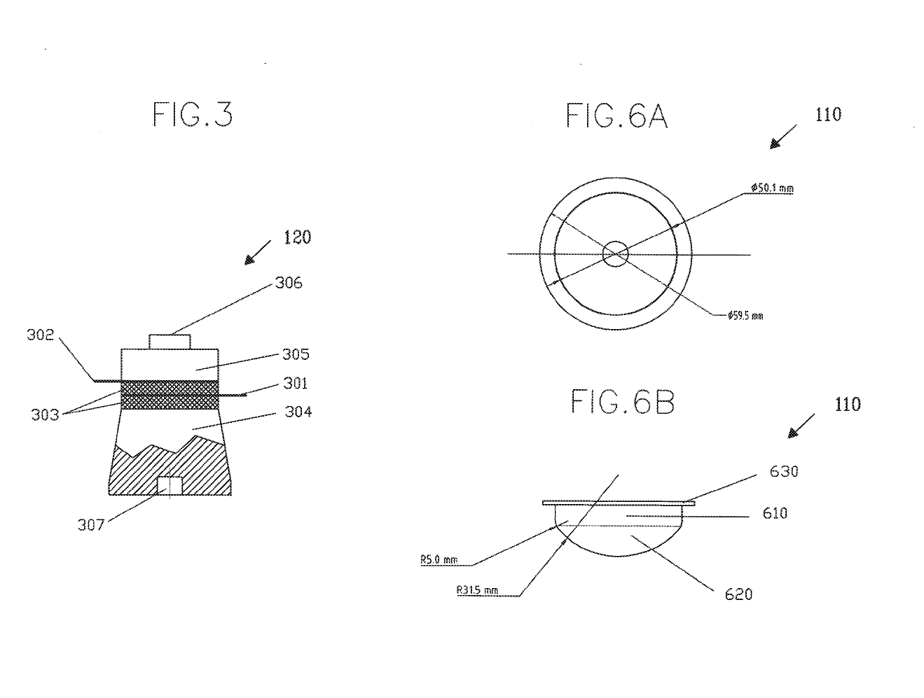

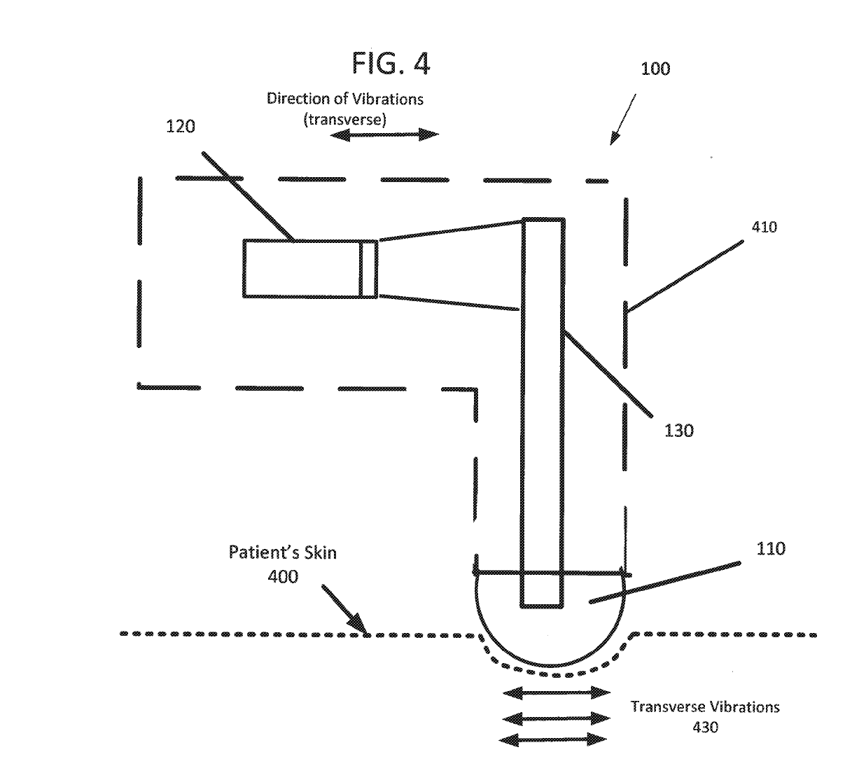

In one embodiment, the semisphere portion 110 of the probe 100 has a radius of 20 mm, so that transverse ultrasound vibrations are applied to a depth of 20 mm by way of pressing the semisphere portion of the probe 100 against the patient's skin during treatment of the patient's skin to damage and/or destroy adipose cells underneath the patient's skin. FIG. 4 shows the probe 100 according to an embodiment being pressed against the patient's skin 400, so that the semisphere portion 110 of the probe 100 is positioned 20 mm inward with respect to upper surfaces of the skin that are adjacent to but not in direct contact with the semisphere portion 110 of the probe 100. In FIG. 4, the housing of the probe 100 is shown by way of the dashed line region 410, whereby the components within the housing of the probe 100 that create the transverse ultrasound vibrations and provide those vibrations to the skin 400 are shown by way of the ultrasound transducer 120 and the connector rod 130. Also, the direction of transverse ultrasound waves to be applied to the patient's skin 410 are shown by way of double-ended arrows 430.

In some embodiments, the strength of the transverse ultrasound vibrations is strong enough such that adipose cells located within a range up to 40 mm beneath the patient's skin are damaged and/or destroyed when subjected to those transverse ultrasound vibrations.

By providing an apparatus for directly applying transverse ultrasound waves output from an ultrasound transducer provided within a probe to a patient's skin, a less complex, easier-to-manufacture, and less-susceptible-to-malfunction probe than what is described in the Britva patent is obtained, and whereby the inventor of this application has determined that supplying only transverse ultrasound waves to a patient's skin provides a better effect than applying both transverse and longitudinal ultrasound waves at a same time period or at consecutive time periods.

Also, by utilizing a semisphere portion having a smooth outer surface at a distal end of the probe, whereby no ridges or undulations are provided on the outer surface of the semisphere portion (in contrast to the structure of Britva), a smoother treatment effect can be obtained, whereby the semisphere portion can easily glide over a portion of the patient's skin to be treated to damage and/or destroy adipose cells located beneath that portion of the patient's skin. Also, since there is no need to have a complex-shaped distal portion of the probe as required in Britva's structure to convert longitudinal vibrations to transverse vibrations, an easier-to-manufacture probe can be obtained, and as stated above, a more pleasant effect can be obtained during treatment of a patient's skin due to the smooth (e.g., non-undulating and non-ridged) shape of the semisphere portion of the probe that is in direct contact with the patient's skin.

The probe 100 of FIG. 1 includes a main body portion 105 that houses an ultrasound transducer 120 as seen in FIG. 5, in which the main body portion 105 is provided at a proximal end of the probe 100 furthest from the patient's skin when the patient is being treated with the probe 100. The probe 100 further includes a semisphere portion 110 provided at a distal end of the probe 100 that is configured to contact the patient's skin when the patient is being treated with the probe 100. The probe 100 further includes an intermediate portion 107 provided between the main body portion 105 and the semisphere portion 110, in which the intermediate portion 107 is disposed substantially perpendicular to the main body portion 105 such that a main axis of the main body portion 105 is provided along a first plane 140 substantially parallel to a second plane 150 corresponding to a surface of the patient's skin being treated with the probe 100, and such that a main axis of the intermediate portion 107 is provided along a third plane 160 substantially perpendicular to the second plane.

The ultrasound transducer is configured to vibrate along the first plane 140 and to thereby cause the semisphere portion 110 to vibrate substantially parallel to the patient's skin due to a connector provided within the intermediate portion that connects the ultrasound transducer to the semisphere. This results in transverse ultrasound waves being applied to the patient's skin by way of the probe 100, which results in destruction and/or damage to adipocytes located beneath a dermis of the patient's skin.

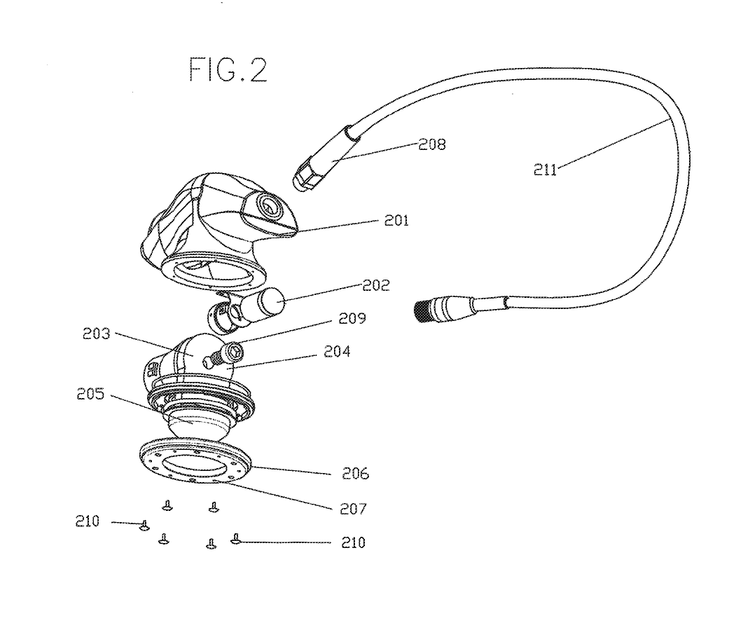

FIG. 2 shows in exploded view various components that may be utilized to create a probe 100 according to one or more embodiments. The components of the probe 100 may include and upper plastic housing 201, an optional electric motor 202, an ultrasound transducer 203, a connector rod 204, a semisphere portion 205 attached to the connector rod 204, a lower plastic housing 206, optional metal electrodes 207, an electric cable inlet housing 208, a connector rod screw 209, lower plastic housing screws 210, and electrical cable 211. The electric motor 202 may be provided with an eccentric in order to generate additional acoustic waves, such as between 50-100 Hz as per dermoelectroporation technology. The metal electrodes 207 may be used to apply additional electric current to the patient's skin, according to the dermoelectroporation technology.

FIG. 3 shows details of an ultrasound transducer 120 that can be provided within the main body portion 105 of the probe 100, according to one or more embodiments. The ultrasound transducer 120 may be constructed as a piezoelectric device or other type of device that provides ultrasound vibrations. In some configurations, two piezoelectric plates are coupled to each other, with one plate causing a vibration in an opposite direction with respect to the other plate. According to one or more embodiments, the ultrasound transducer (having a total length of 57 mm, a minimum width of 38 mm, and a maximum width of 48 mm) may include a first piezoelectric crystal connection 301, a second piezoelectric crystal connection 302, a piezoelectric crystal 303, a front metal part 304, which may have a maximum width of 48 mm, a back metal part 305, which may have a width of 38 mm, and a length of 23.5 mm, screw 306, which may keep parts connected and having a top part that may extend outward from the back metal part 305 by 5.5 mm, and a hole 307 for receiving the screw 209 of the connector rod 204, which may be approximately 7 mm long by 10 mm wide. In some embodiments, the semispherical portion may be configured to press into the surface of the skin at a depth of between 5 mm to 40 mm thus generating transverse ultrasound waves at the corresponding depth. In some embodiments, the diameter of the semispherical portion may be approximately 50 mm.

With reference to FIG. 1 and FIG. 5, the ultrasound transducer 120 is housed in the main body portion 105 of the probe 100, which has a main axis 140 that is substantially parallel to the patient's skin to be treated by the probe. With such a construction, the ultrasound transducer 120 outputs vibrations that are substantially parallel to the patient's skin, and thus are transverse ultrasound vibrations.

The transverse ultrasound vibrations output from the ultrasound transducer are transferred to the semisphere portion of the probe by way of a metal plate, or connector rod 130, as shown in FIG. 5. The connector rod 130 may be configured as having one main axis 510, and thus corresponding to a long rod-shaped structure. The connector rod 130 may be of an aluminum construction (e.g., an aluminum plate) or of another lightweight metal construction. One end of the connector rod 130 is in direct contact to the ultrasound transducer 120 and directly receives the transverse ultrasound waves output from the ultrasound transducer 120. The other end of the connector rod 130 is in direct contact with the semisphere portion 110 of the probe 100, and thus transfers the transverse ultrasound waves output from the ultrasound transducer 120 directly to the semisphere portion 110 of the probe 100, and thus directly to the patient's skin in contact with the semisphere portion 110 of the probe 100. With reference to FIG. 1 and FIG. 5, the connector rod 130 is housed primarily in the main body portion 105 and the intermediate portion 107 of the probe, whereby a proximal end of the connector rod is housed within the main of of the probe 100 and whereby a distal end of the connector rod 130 is housed within the semisphere portion 110 of the probe 100.

In some embodiments, to maintain as lightweight a construction as possible, the connector rod 130 has many holes provided along its main axis, as does the semisphere portion 110 of the probe 100. Also, due to the fact that the speed of ultrasound vibrations traveling on metal, such as aluminum, is about five (5) times the speed of ultrasound waves traveling on a patient's skin, the metal connector rod 130 can be considered to be rigid as compared to the patient's skin. This is also the case with respect to the semisphere portion 110 of the probe 100 that is connected to the connector rod 130, which can also be considered to be rigid with respect to the patient's skin. Due to the holes provided along the connector rod 130 and along the outer surface of the semisphere portion 110, the connector rod/semisphere structure has a mass weight less than the mass weight of the skin that it is to drive with transverse ultrasound vibrations. This provides an optimal way to apply transverse ultrasound vibrations to the patient's skin, so as to achieve a good effect for damaging and/or destroying adipose cells under the patient's skin (e.g., between 2 to 40 mm under the dermis of the skin). The holes provided on the outer surface of the semisphere portion 110 may be in the range of from 0.01 to 0.1 mm, so that they do not cause any discomfort when the semisphere portion 110 is slid over a portion of the patient's skin to be treated by way of the probe 100.

Due to the semisphere portion 110 having a radius of 20 mm in some embodiments, the first 20 mm under the patient's skin are subject to the transverse ultrasound vibrations as the semisphere portion 110 of the probe 100 is pressed against the patient's skin 400, as shown in FIG. 4. These transverse ultrasound vibrations have a maximum intensity at around 20 mm under the skin surface (for a semisphere portion 110 having a 20 mm radius), which is determined by the inventor to be an optimal depth for damaging and destroying adipocytes under the skin surface.

In other embodiments, the semisphere portion 110 of the probe 100 has a different size, such as between 15 mm to 25 mm, whereby similar positive effects by damaging and destroying adipocytes under the skin surface are obtained for such structures.

FIGS. 6A and 6B respectively show a front view and a side view of the semisphere portion of the probe, according to one or more embodiments. By way of example and not by way of limitation, the semisphere portion 110 includes a flat-sided base portion 610 that is of 8 mm in depth, and a curved portion 620 that is of 12 mm in depth with respect to a point of the curved portion 620 farthest from the base portion 610. By way of example and not by way of limitation, the semisphere portion 110 has a thin plate portion 630 of 1.5 mm, for attachment to the intermediate portion 107 of the probe (see FIGS. 1 and 5, for example). The attachment of the thin plate portion 630 (and thus the semisphere portion 110) to the rest of the probe 100 may be by way of screws or other fixation devices (see screws 210 in FIG. 2, for example). In the embodiment as shown in FIG. 6A, the thin plate portion 630 is of a circular shape and has a diameter of 59.5 mm, and the flat-sided base portion 610 and the curved portion 620 of the semisphere 110 have a diameter of 50.1 mm. According to one embodiment, the curvature radius of the semisphere portion 110 is 31.52 mm, and the curvature radius of the part that connects the semisphere portion 110 to the 8 mm (in length) cylinder is 5 mm. Other curvature radiuses can be utilized, while keeping within the spirit and scope of the invention as described herein.

By having a smooth shaped semisphere portion 110 of the probe 100 that is in direct contact with a skin surface to the treated, a good massaging effect can be obtained to the patient at the same time adipose cells are damaged and destroyed beneath the patient's skin. This dual benefit provides for a pleasant treatment experience for removing fat cells underneath a patient's skin. In some embodiments, an oil-based gel or other type of lubricating gel may be applied to the semisphere portion outer surface, to enhance the massaging effect when the semisphere portion is slid across the patient's skin. For example, in some embodiments, a gel-holding region within the semisphere portion 110 of the probe 100 may be included in some embodiments, whereby gel is output from the gel-holding region and through holes on the exterior housing of the semisphere portion 110 of the probe 100, and thereby onto the patient's skin, to enhance the movement of the semisphere portion 110 of the probe on the patient's skin during treatment of the patient. Actuation of a trigger (not shown in the drawings) on the probe 100 by a user of the probe 100 causes expelling of the gel from the gel-holding region, through the holes of the semisphere portion 110 of the probe 100, and thereby onto the patient's skin.

In some embodiments, the probe 100 has its own power supply (not shown in the drawings), such as a battery pack, and in other embodiments, the probe is configured to have an electrical cord that can be connected to an electrical output, to provide the necessary power to the components within the probe 100.

In some embodiments, the transverse ultrasound vibrations are provided in pulses of energy to the patient's skin, such as at a 20% duty cycle. Thus, for an output power of 20-35 watts/cm² output by the ultrasound transducer, the average power applied to the patient's skin at a 20-50% duty cycle is about 1-7 watts/cm², thereby providing a power flux to the patient's skin of 1-7 watts/cm², which does not cause much if any discomfort to the patient during treatment of the patient's skin.

In some embodiments, the ultrasound frequency of the transverse ultrasound vibrations is 32 kHz, and in other embodiments the ultrasound frequency of the transverse ultrasound vibrations is a frequency in the range of from 20-60 kHz.

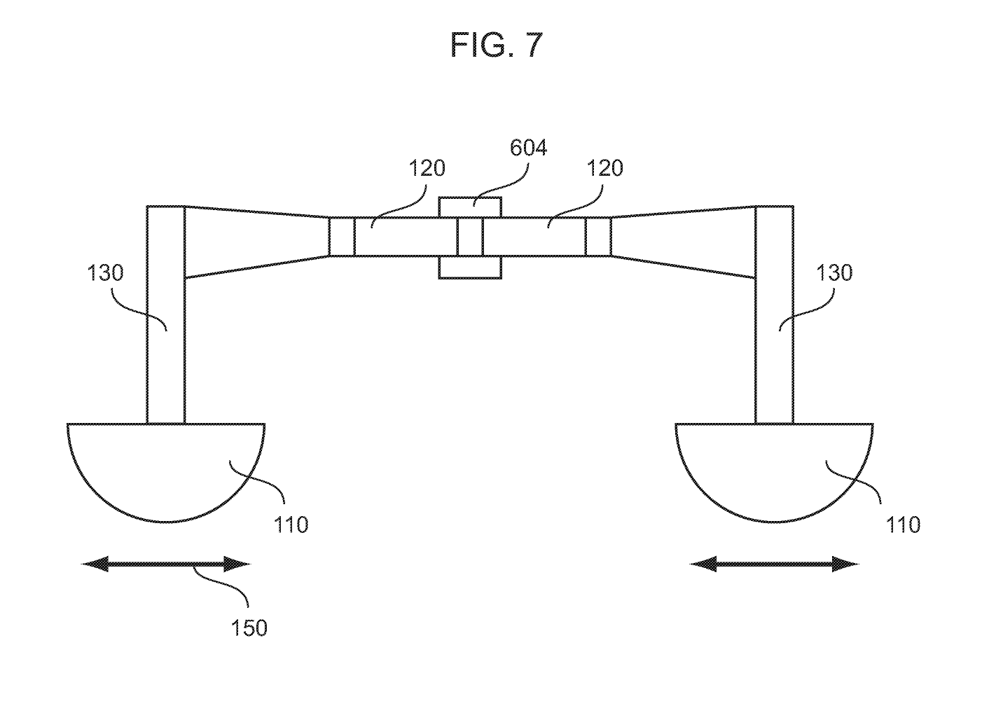

FIG. 7 shows an arrangement in which a device having two semisphere portions 110 is provided. In some embodiments, the arrangement may be constructed using two transducer mechanisms 100 coupled by a clamp 604. The two semisphere portions 110 may be coupled respectively via connector rods 130 to respective ultrasound transducers 120. Because of the arrangement, the two semisphere portions 110 vibrate in opposition of phase such that when the direction of vibration of the left devices is toward left, the direction of vibration of right device is toward right. The effect may be achieved by driving each of the two ultrasound transducers 120, which may be piezo electric transducers, with the same electrical signal. By arranging the two semisphere portions 110 and driving the two ultrasound transducers 120 as described above, any vibration of the semisphere portions 110 in direction perpendicular to the patient's skin may be completely avoided. In other words, by the disclosed arrangement, the generation of unwanted longitudinal ultrasound waves, e.g. waves that penetrate deeper than desired, in direction perpendicular to the skin is avoided.

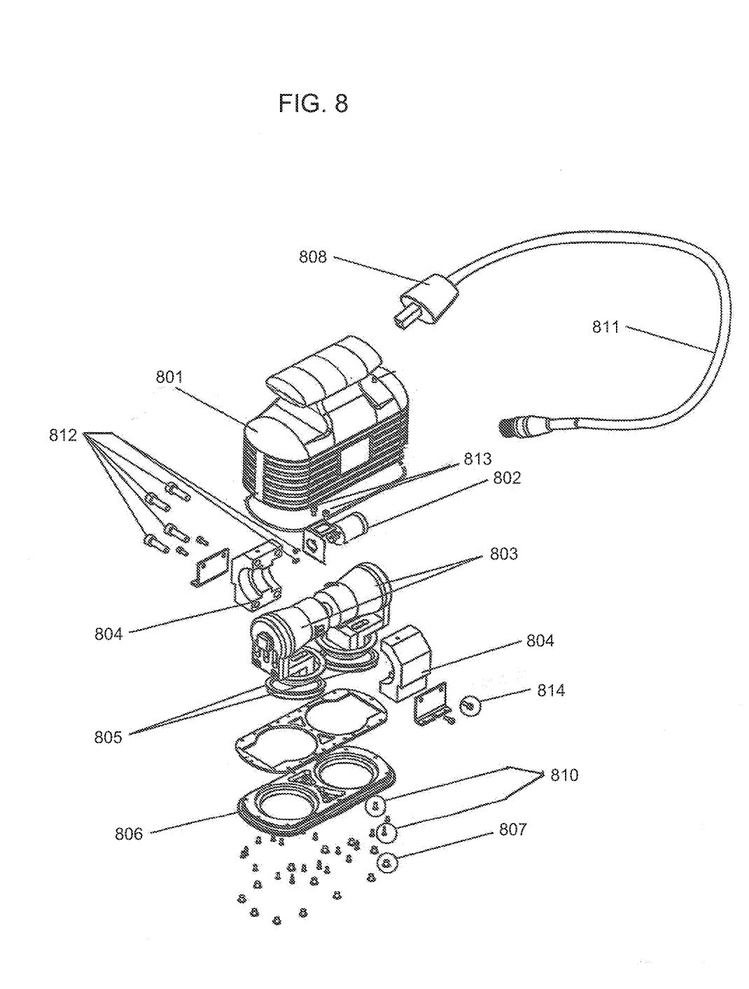

FIG. 8 shows in exploded view various components that may be utilized to create a probe according to one or more embodiments. The components of the probe may include and upper plastic housing 801, an optional electric motor 802, an ultrasound transducer 803, a clamp 804, a pair of semisphere portions 805 attached via a connector rod, a lower plastic housing 806, optional metal electrodes 807, an electric cable inlet housing 808, connector rod screws, lower plastic housing screws 810, and electrical cable 811 and various screws 812-814. Each of the electric motors 802 may be provided with an eccentric in order to generate additional acoustic waves, such as between 50-100 Hz as per dermoelectroporation technology. The metal electrodes 807 may be used to apply additional electric current to the patient's skin, according to the dermoelectroporation technology. Where not specifically mentioned, the characteristics of the ultrasound transducers and probes may be the same as, for example, the probe 100 discussed in connection with FIG. 2 and therefore a duplicate description will be omitted for brevity.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order shown, or sequential order, to achieve desirable results.

What is claimed is:

1. An apparatus for treating adipose tissue located beneath a skin surface of a patient comprising:
a main body portion that houses a first ultrasound transducer positioned in such a way in order to vibrate only in a first direction parallel to the skin surface;
a first semispherical portion provided at first distal end of the apparatus and configured to be pressed against the skin in order to bend the skin in such a way that a lower portion of the first semispherical portion is positioned at a same depth as a surrounding first layer of adipocytes to be treated; and
a first rigid intermediate portion provided between the first ultrasound transducer and the first semispherical portion, the first rigid intermediate portion coupling vibration from the first ultrasound transducer to the first semispherical portion to cause the first semispherical portion to vibrate only in the first direction, with a same intensity and with a same phase of the first ultrasound transducer and only parallel to the skin surface and without vibration in a direction perpendicular to skin surface, the coupled vibration generating transverse ultrasound waves perpendicular to the skin surface and at a predetermined depth, and longitudinal waves in the first direction parallel to the skin surface; wherein:
a cross-sectional curvature radius of a distal end of the first semispherical portion is set to establish a depth at which adipose cells in the adipose tissue located beneath the skin surface of the patient are damaged or destroyed when the first semispherical portion is pressed against the skin surface of the patient to the established depth; and
the cross-sectional curvature radius of the distal end of the first semispherical portion is further set such that a maximum intensity of the transverse ultrasound waves occurs at the established depth.

2. The apparatus according to claim 1, wherein the first semispherical portion has a smooth outer surface to facilitate movement on the skin surface during pressing.

3. The apparatus according to claim 1, wherein a frequency of the transverse ultrasound waves is within the range of from 20 kHz to 60 kHz.

4. The apparatus according to claim 3, wherein the transverse ultrasound waves are pulsed within a range of 50 Hz to 100 Hz and the duty cycle between 10% and 50%.

5. The apparatus of claim 1, wherein the first semispherical portion is configured to press into the surface of the skin at a depth of between 5 mm to 40 mm thus generating transverse ultrasound waves at the corresponding depth.

6. The apparatus according to claim 1, wherein a diameter of the first semispherical portion is approximately 50 mm.

7. The apparatus according to claim 3, wherein a power flux applied to patient skin is in the range between 1-3 W/cm².

8. The apparatus according to claim 1, wherein the intermediate portion between the transducer and the semispherical portion is made of aluminum.

9. The apparatus according to claim 1, wherein the intermediate portion between the transducer and the semispherical portion is made of steel.

10. The apparatus according to claim 1, further comprising:
a second ultrasound transducer housed in the main body portion and positioned next to and in an opposing driving relation to the first ultrasound transducer in such a way as to vibrate only in a second direction parallel to the skin surface and opposite to the first direction;
a second semispherical portion provided at the distal end of the apparatus and configured to be pressed against the skin in order to bend the skin in such a way that a lower portion of the second semispherical portion is positioned at the same depth as the surrounding first layer of the adipocytes to be treated; and
a second rigid intermediate portion provided between the second ultrasound transducer and the second semispherical portion, the second rigid intermediate portion coupling vibration from the second ultrasound transducer to the second semispherical portion to cause the second semispherical portion to vibrate only in a same direction, with a same intensity and with a same phase of the second ultrasound transducer; in an opposite direction, with a same intensity and with an opposite phase of the first ultrasound transducer and only parallel to the skin surface and without vibration in the direction perpendicular to the skin surface, the coupled vibration generating transverse ultrasound waves perpendicular to the skin surface and at the predetermined depth, and longitudinal waves in the direction parallel to the skin surface, wherein:
a cross-sectional curvature radius of a distal end of the second semispherical portion is set at the depth at which adipose cells in the adipose tissue located beneath the skin surface of the patient are damaged or destroyed when the second semispherical portion is pressed against the skin surface of the patient to the established depth; and
the cross-sectional curvature radius of the distal end of the second semispherical portion is further set such that the maximum intensity of the transverse ultrasound waves occurs at the established depth.

11. A method for treating adipose tissue located beneath a skin surface of a patient, comprising:
outputting, using a first ultrasound transducer, transverse ultrasound waves in a first direction parallel to the skin surface; and
generating longitudinal ultrasound waves, wherein:
the longitudinal ultrasound waves are generated by the first ultrasound transducer parallel to the skin connected by a first rigid intermediate portion to a first semispherical portion configured to be pressed to the skin surface such that a lower portion of the first semispherical portion in contact with the skin surface is positioned at a same depth as a first layer of adipocytes under the skin surface;
a cross-sectional curvature radius of a distal end of the first semispherical portion is set to establish a depth at which the adipocytes are damaged or destroyed when the first semispherical portion is pressed against the skin surface of the patient to the established depth; and
the cross-sectional curvature radius of the distal end of the first semispherical portion is further set such that a maximum intensity of the of the transverse ultrasound waves occurs at the established depth.

12. The method according to claim 11, wherein a frequency of the transverse ultrasound waves is within a range of from 20 KHz to 60 KHz.

13. The method according to claim 11, wherein the first rigid intermediate portion comprises an aluminum rod.

14. The method according to claim 11, wherein the first ultrasound transducer is a piezoelectric device.

15. The method according to claim 11, wherein the transverse ultrasound waves are output at a duty cycle between 10%-50%.

16. The method according to claim 14, wherein an output power of the first ultrasound transducer applied to the skin surface is in a range of between 1-3 watts/cm².

17. The method according to claim 11, further comprising:
outputting, using a second ultrasound transducer, transverse ultrasound waves in a second direction parallel to the skin surface and opposite the first direction; and
generating longitudinal ultrasound waves, wherein:
the longitudinal ultrasound waves are generated by the second ultrasound transducer parallel to the skin connected by a second rigid intermediate portion to a second semispherical portion configured to be pressed to the skin surface such that a lower portion of the second semispherical portion in contact with the skin surface is positioned at a same depth as a first layer of adipocytes under the skin surface;
a cross-sectional curvature radius of a distal end of the second semispherical portion is set to the depth at which the adipocytes are damaged or destroyed when the second semispherical portion is pressed against the skin surface of the patient to the established depth; and
the cross-sectional curvature radius of the distal end of the second semispherical portion is further set such that the maximum intensity of the of the transverse ultrasound waves occurs at the established depth.
